(19) United States
(12) Patent Application Publication
(10) Pub. No.: US 2022/0039428 A1
(43) Pub. Date: Feb. 10, 2022

(54) METHOD TO IMPROVE SOLUBILITY OF DOUBLE HYDROPHOBIC PROTEINS IN WATER

(71) Applicant: Jiangnan University, Wuxi (CN)

(72) Inventors: Ren WANG, Wuxi (CN); Tao WANG, Wuxi (CN); Wei FENG, Wuxi (CN); Zhengxing CHEN, Wuxi (CN); Lulu LI, Wuxi (CN); Fangsi LI, Wuxi (CN)

(21) Appl. No.: 17/234,762

(22) Filed: Apr. 19, 2021

(30) Foreign Application Priority Data
Aug. 5, 2020 (CN) 202010777008.4

(51) Int. Cl. A23J 3/14; C07K 1/14

(52) U.S. Cl. CPC A23J 3/14; C07K 1/145

(57) ABSTRACT

The disclosure provides a method to improve the solubility of double hydrophobic proteins in water. The method includes: 1) adding a first hydrophobic protein and a second hydrophobic protein to distilled water, stirring, adding an aqueous alkali to the mixture until the pH of the mixture is greater than or equal to 10.0; 2) stirring the mixture for 30-120 min at 500-2000 rpm; 3) stirring the mixture obtained from 2) for 45-75 min at 500-2000 rpm and meanwhile dropwise adding an acid solution to the mixture until the pH of the mixture is 7.0, to yield a first solution; 4) dialyzing the first solution of the acid solution and the mixture in 3) for 20-30 hours, to yield a second solution; 5) centrifuging the second solution obtained from 4) at 4000-10000×g for 10-30 min, and collecting a supernatant; and 6) freezing and drying the supernatant.

### CROSS-REFERENCE TO RELATED APPLICATIONS

Pursuant to 35 U.S.C.§ 119 and the Paris Convention Treaty, this application claims foreign priority to Chinese Patent Application No. 202010777008.4 filed on Aug. 5, 2020, the contents of which, including any intervening amendments thereto, are incorporated herein by reference. Inquiries from the public to applicants or assignees concerning this document or the related applications should be directed to: Matthias Scholl PC., Attn.: Dr. Matthias Scholl Esq., 245 First Street, 18th Floor, Cambridge, Mass. 02142.

### BACKGROUND

The disclosure relates to a method to improve the solubility of double hydrophobic proteins in water.

Conventional solubilization technologies of hydrophobic proteins include enzymatic hydrolysis, chemical modification and physical modification.

Excessive enzymatic hydrolysis decreases the nutritional value and destroys functional properties of proteins, while mild enzymatic hydrolysis may fail to achieve the solubilization effect.

Chemical modification is to modify the protein group through a chemical reagent. However, chemical reactions may lead to the destruction of protein structure and reduce its nutritional value.

Physical modification is often used in combination with enzymatic hydrolysis and chemical modification, which is energy-consuming and involves expensive equipment.

### SUMMARY

The disclosure provides a method to improve the solubility of double hydrophobic proteins in water, the method comprising:
1) adding a first hydrophobic protein and a second hydrophobic protein in a mass ratio of 1:0.1-1:10 to distilled water, stirring, adding an aqueous alkali to a mixture of the first hydrophobic protein, the second hydrophobic protein, and the distilled water until a pH of the mixture is greater than or equal to 10.0;
2) stirring the mixture for 30-120 min at 500-2000 rpm to expose secondary structures of the first hydrophobic protein and the second hydrophobic protein;
3) stirring the mixture obtained from 2) for 45-75 min at 500-2000 rpm and meanwhile dropwise adding an acid solution to the mixture until the pH of the mixture is 7.0, to yield a first solution, so that the first hydrophobic protein is structurally connected to the second hydrophobic protein through hydrophobic groups of the secondary structures;
4) dialyzing the first solution of the acid solution and the mixture in 3) for 20-30 hours, to yield a second solution;
5) centrifuging the second solution obtained from 4) at 4000-10000×g for 10-30 min, and collecting a supernatant; and
6) freezing and drying the supernatant, to yield a water-soluble hydrophobic protein complex.

In a class of this embodiment, the first hydrophobic protein is rice protein, and the second hydrophobic protein is hemp protein or walnut protein.

In a class of this embodiment, the first hydrophobic protein and the second hydrophobic protein form the water-soluble hydrophobic protein complex through the hydrophobic groups of the secondary structures of the first hydrophobic protein and the second hydrophobic protein.

The following advantages are associated with the method to improve the solubility of double hydrophobic proteins in water:

1. The method of the disclosure is implemented in mild conditions, and the reagents used are all food additives, and there is no risk of chemical reagent residues.

2. The method is novel because the modified sites are precisely located in the secondary structure of hydrophobic proteins. First, the secondary structures of hydrophobic proteins are fully exposed by in an alkali solution; second, the secondary structures of two kinds of hydrophobic proteins are combined through hydrophobic groups after acid neutralization; thereafter, under neutral condition, the two kinds of hydrophobic protein form a complex, of which the hydrophobic region is fully embedded, while the hydrophilic region is exposed, thus obtaining a stable aqueous solution of proteins. As shown in FIG. 2, the main subunit structures of the hydrophobic proteins treated by the method of the disclosure are completely maintained, and the primary structure is not destroyed. This shows that the amino acids of the two proteins are complete, so that their nutritional and functional properties are unaffected.

### BRIEF DESCRIPTION OF THE DRAWING

FIG. 1 is a sodium dodecyl sulfate-polyacrylamide gel electrophoresis (SDS-PAGE) diagram of precipitates though centrifugation in Examples 1-3 of the disclosure;

FIG. 2 is a sodium dodecyl sulfate-polyacrylamide gel electrophoresis (SDS-PAGE) diagram of the prepared water-soluble hydrophobic protein complex products in Examples 1-3 of the disclosure; and

FIG. 3 shows rice protein, hemp protein, and a water-soluble hydrophobic protein complex prepared in Example 3 of the disclosure.

### DETAILED DESCRIPTION

To further illustrate, embodiments detailing a method to improve the solubility of double hydrophobic proteins in water are described below. It should be noted that the following embodiments are intended to describe and not to limit the disclosure.

Unless otherwise specified, the experimental methods used in the following examples are conventional methods.

Unless otherwise specified, the materials and reagents used in the following examples are available from commercial channels.

In the disclosure, the solubility of rice protein is characterized by the content of soluble rice protein, that is, the mass of rice protein of the water-soluble hydrophobic protein complex accounting for that of rice protein in the raw materials. The mass m₀ of rice protein in the raw materials and the mass m₁ of rice protein in soluble hydrophobic protein complex are both determined by Kjeldahl method.

Spherically, the solubility of rice protein is characterized as follows:

Solubility of rice protein in the water-soluble hydrophobic protein complex (%)=m₁/m₀×100%;

m₀: mass of rice protein in the raw materials; and

m₁: mass of rice protein in the water-soluble hydrophobic protein complex.

The characterization of the solubility of hemp protein and walnut protein is the same as that of rice protein.

### Example 1

Provided is a method to improve the solubility of double hydrophobic proteins in water, the method comprising:

1) adding rice protein and hemp protein in a mass ratio of 1:0.2 to distilled water, stirring, adding an aqueous alkali to a mixture of the rice protein, the hemp protein, and the distilled water until a pH of the mixture is equal to 10.0;

2) stirring the mixture for 60 min at 600 rpm to expose secondary structures of the rice protein and the hemp protein;

3) stirring the mixture obtained from 2) for 60 min at 900 rpm and meanwhile dropwise adding an acid solution to the mixture until the pH of the mixture is 7.0, to yield a first solution, so that the rice protein is structurally connected to the hemp protein through hydrophobic groups of the secondary structures;

4) dialyzing the first solution of the acid solution and the mixture in 3) for 22 hours, to yield a second solution;

5) centrifuging the second solution obtained from 4) at 8000×g for 20 min, and collecting a supernatant; and

6) freezing and drying the supernatant, to yield a water-soluble hydrophobic protein complex.

### Example 2

Provided is a method to improve the solubility of double hydrophobic proteins in water, the method comprising:

1) adding rice protein and walnut protein in a mass ratio of 1:0.5 to distilled water, stirring, adding an aqueous alkali to a mixture of the rice protein, the walnut protein, and the distilled water until a pH of the mixture is equal to 11.0;

2) stirring the mixture for 100 min at 900 rpm to expose secondary structures of the rice protein and the walnut protein;

3) stirring the mixture obtained from 2) for 45 min at 900 rpm and meanwhile dropwise adding an acid solution to the mixture until the pH of the mixture is 7.0, to yield a first solution, so that the rice protein is structurally connected to the walnut protein through hydrophobic groups of the secondary structures;

4) dialyzing the first solution of the acid solution and the mixture in 3) for 24 hours, to yield a second solution;

5) centrifuging the second solution obtained from 4) at 10000×g for 10 min, and collecting a supernatant; and

6) freezing and drying the supernatant, to yield a water-soluble hydrophobic protein complex.

### Example 3

Provided is a method to improve the solubility of double hydrophobic proteins in water, the method comprising:

1) adding rice protein and hemp protein in a mass ratio of 1:1 to distilled water, stirring, adding an aqueous alkali to a mixture of the rice protein, the hemp protein, and the distilled water until a pH of the mixture is equal to 12;

2) stirring the mixture for 45 min at 1200 rpm to expose secondary structures of the rice protein and the hemp protein;

3) stirring the mixture obtained from 2) for 75 min at 600 rpm and meanwhile dropwise adding an acid solution to the mixture until the pH of the mixture is 7.0, to yield a first solution, so that the rice protein is structurally connected to the hemp protein through hydrophobic groups of the secondary structures;

4) dialyzing the first solution of the acid solution and the mixture in 3) for 26 hours, to yield a second solution;

5) centrifuging the second solution obtained from 4) at 5000×g for 30 min, and collecting a supernatant; and

6) freezing and drying the supernatant, to yield a water-soluble hydrophobic protein complex.

### Example 4

The properties of the water-soluble hydrophobic protein complex products prepared in Examples 1-3 are shown in Table 1.

TABLE 1

| | Raw materials | | | Example 1 | | Example 2 | | Example 3 | |
|---|---|---|---|---|---|---|---|---|---|
| | Rice | Hemp | Walnut | Rice | Hemp | Rice | Walnut | Rice | Hemp |
| | protein | protein | protein | protein | protein | protein | protein | protein | protein |
| Content of water- | 9.68 | 26.47 | 25.84 | 87.06 | 100.00 | 90.74 | 100.00 | 96.45 | 100.00 |
| soluble protein | | | | | | | | | |
| (%) | | | | | | | | | |
| Times of increase | / | / | / | 8.99 | 3.78 | 9.37 | 3.87 | 9.96 | 3.78 |

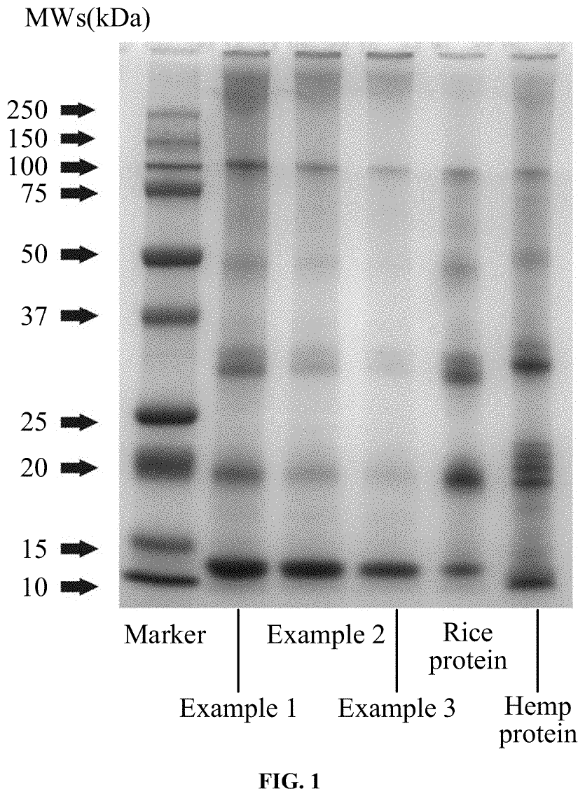

As shown in FIG. 1, during the preparation of the soluble hydrophobic protein complex, the main component of the precipitate is the rice protein, showing that the hemp protein and the walnut protein are completely dissolved in this process.

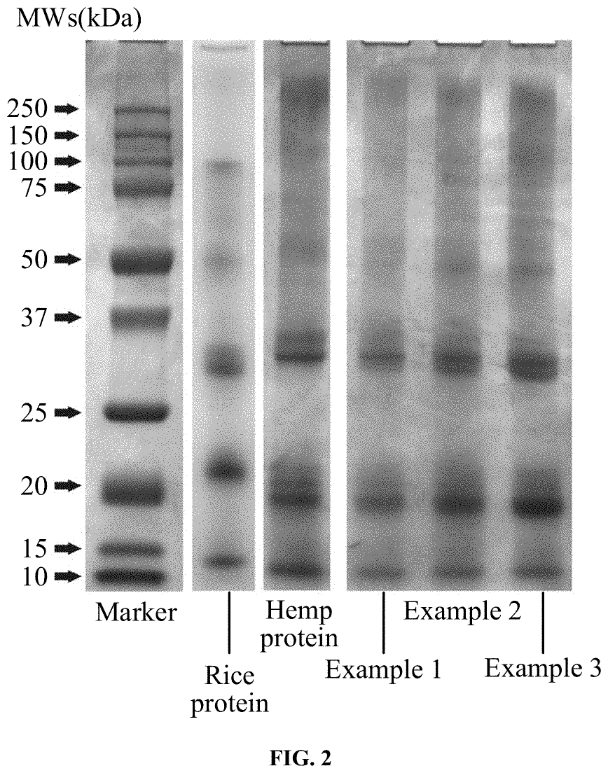

As shown in FIG. 2, during the preparation of the soluble hydrophobic protein complex, the obtained supernatant contains all subunits of the rice protein, hemp protein and walnut protein, showing that the primary structures of the proteins are completely maintained in this process.

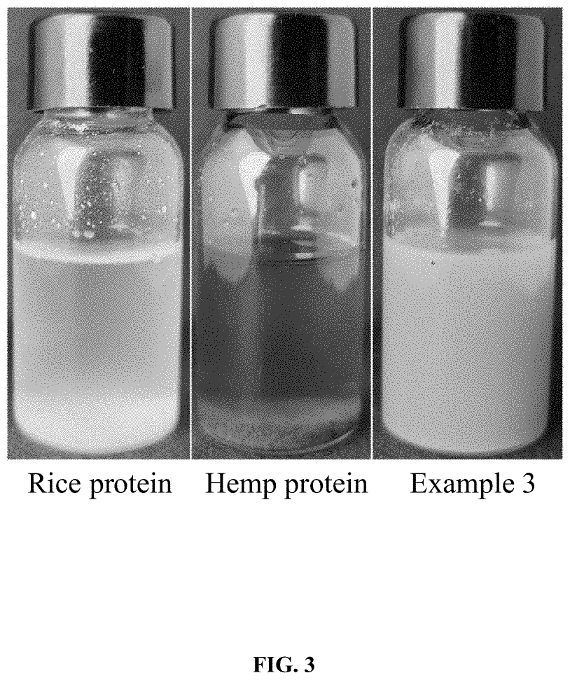

As shown in FIG. 3, with rice protein or hemp protein as a raw material alone, after being treated by the method of the disclosure, the solubility of the proteins is not improved. However, after being treated together, the two proteins are stably dispersed in water.

It will be obvious to those skilled in the art that changes and modifications may be made, and therefore, the aim in the appended claims is to cover all such changes and modifications.

What is claimed is:

1. A method, comprising:
1) adding a first hydrophobic protein and a second hydrophobic protein in a mass ratio of 1:0.1-1:10 to distilled water, stirring, adding an aqueous alkali to a mixture of the first hydrophobic protein, the second hydrophobic protein, and the distilled water until a pH of the mixture is greater than or equal to 10.0;
2) stirring the mixture for 30-120 min at 500-2000 rpm to expose secondary structures of the first hydrophobic protein and the second hydrophobic protein;
3) stirring the mixture obtained from 2) for 45-75 min at 500-2000 rpm and meanwhile dropwise adding an acid solution to the mixture until the pH of the mixture is 7.0, to yield a first solution, so that the first hydrophobic protein is structurally connected to the second hydrophobic protein through hydrophobic groups of the secondary structures;
4) dialyzing the first solution of the acid solution and the mixture in 3) for 20-30 hours, to yield a second solution;
5) centrifuging the second solution obtained from 4) at 4000-10000×g for 10-30 min, and collecting a supernatant; and
6) freezing and drying the supernatant, to yield a water-soluble hydrophobic protein complex.

2. The method of claim 1, wherein the first hydrophobic protein is rice protein, and the second hydrophobic protein is hemp protein or walnut protein.

3. The method of claim 1, wherein the first hydrophobic protein and the second hydrophobic protein form the water-soluble hydrophobic protein complex through the hydrophobic groups of the secondary structures of the first hydrophobic protein and the second hydrophobic protein.
